(19) United States
(12) Patent Application Publication
(10) Pub. No.: US 2017/0150919 A1
(43) Pub. Date: Jun. 1, 2017

(54) BREATH DETECTION DEVICE AND OPERATING METHOD THEREOF

(71) Applicant: PIXART IMAGING INC., Hsin-Chu County (TW)

(72) Inventors: Chih-Yuan CHUANG, Hsin-Chu County (TW); Wei-Ru HAN, Hsin-Chu County (TW); Ming-Chang LI, Hsin-Chu County (TW)

(21) Appl. No.: 15/337,614

(22) Filed: Oct. 28, 2016

(30) Foreign Application Priority Data
Dec. 1, 2015 (TW) 104140129
Jan. 26, 2016 (TW) 105102395

(51) Int. Cl. A61B 5/00; A61B 5/08

(52) U.S. Cl. CPC A61B 5/486; A61B 5/0059; A61B 5/0816; A61B 5/7278; A61B 5/0002; A61B 5/742; A61B 5/725; A61B 5/746; A61B 5/7455; A61B 5/7405; A61B 5/6802

(57) ABSTRACT

A breath detection method includes the steps of: receiving a PPG signal; recognizing a low frequency carrier of the PPG signal; recognizing a rising part of the low frequency carrier and a falling part of the low frequency carrier, wherein a frequency of the low frequency carrier represents a breathing cycle period of a user, the rising part represents one of a breathing out state and a breathing in state of the user, and the falling part represents the other one of the breathing out state and the breathing in state; and real-timely outputting at least one of the breathing cycle period, the breathing out state or the breathing in state.

### CROSS REFERENCE TO RELATED APPLICATION

This application claims the priority benefit of Taiwan Patent Application Serial Number 104140129, filed on Dec. 1, 2015, and Taiwan Patent Application Serial Number 105102395, filed on Jan. 26, 2016, the full disclosures of which are incorporated herein by reference.

### BACKGROUND

1. Field of the Disclosure

This disclosure generally relates to a breath detection device and an operating method thereof that obtain a breathing cycle period of a user using a photoplethysmography (PPG) signal, more particularly, to that capable of real-timely obtaining, by analyzing a PPG signal, information related to the breathing depth and the breathing cycle period from the analyzed PPG signal.

2. Description of the Related Art

So far, there is no scheme capable of real-timely obtaining a breathing cycle period using a PPG signal. In addition, the conventional breath detection device needs at least one minute to be able to obtain a user's respiration rate per minute and is not able to real-timely display a breathing state of each breath such that the application thereof is significantly limited.

### SUMMARY

The present disclosure provides a breath detection device and an operating method thereof that obtain a user's breathing cycle period using a photoplethysmography (PPG) signal.

The present disclosure provides a breath detection device including a light source, a light sensor and a processor. The light source is configured to illuminate a skin surface to make light pass through skin tissues under the skin surface. The light sensor is configured to detect ejected light from the skin tissues to generate a PPG signal. The processor is configured to acquire a low frequency carrier of the PPG signal as a breathing signal.

The present disclosure further provides a breath detection device including a light source, a light sensor, a processor, a display device and a prompt device. The light source is configured to illuminate a skin surface to make light pass through skin tissues under the skin surface. The light sensor is configured to detect ejected light from the skin tissues to generate a PPG signal. The processor is configured to acquire a low frequency carrier of the PPG signal as a breathing signal. The display device is configured to real-timely display a variation curve of the breathing signal changed with time. The prompt device is configured to generate a prompt signal according to a comparison result of comparing intensity, a rising part, a falling part and/or a frequency of the variation curve with at least one threshold.

The present disclosure further provides an operating method of a breath detection device including: obtaining, by a light sensor, a PPG signal from a skin surface; acquiring, by a processor, a low frequency carrier of the PPG signal; identifying, by the processor, a period, a rising part and a falling part of the low frequency carrier, wherein the period represents a breathing cycle period of a user, the rising part represents one of a breathe-out and a breathe-in of the user, and the falling part represents the other one of the breathe-out and the breathe-in of the user; and real-timely outputting at least one of the breathing cycle period, a breathing out state of the breathe-out and a breathing in state of the breathe-in.

### BRIEF DESCRIPTION OF THE DRAWINGS

Other objects, advantages, and novel features of the present disclosure will become more apparent from the following detailed description when taken in conjunction with the accompanying drawings.

FIG. 1 is a photoplethysmography (PPG) signal.

FIG. 2 is a schematic diagram of breathing cycle periods of the PPG signal retrieved from FIG. 1, each period including a rising part and a falling part.

FIGS. 3A and 3B are usage states of a breath detection device according to some embodiments of the present disclosure.

FIG. 4 is a schematic block diagram of a breath detection device according to one embodiment of the present disclosure.

FIG. 5 is a flow chart of an operating method of a breath detection device according to one embodiment of the present disclosure.

### DETAILED DESCRIPTION OF THE EMBODIMENT

It should be noted that, wherever possible, the same reference numbers will be used throughout the drawings to refer to the same or like parts.

Photoplethysmography (PPG) signals are consisted of two parts. When a systole occurs, the blood pressure and blood volume in blood vessels of the whole body have a continuous variation. When a diastole occurs, the blood pressure decreases correspondingly and the blood pumped-out in a previous systole heats the heart valve to cause so-called inflection.

Therefore, a complete PPG waveform includes a mixed effect of said systole and pressures from the blood vessel wall. The PPG signal is obtainable by detecting a volume variation of blood vessels through optical measurements.

To obtain signals related to a user's breathing signal from a PPG signal, it is necessary to obtain the PPG signal at first, and a low frequency carrier of the PPG signal is then identified to determine a corresponding frequency of the low frequency carrier, wherein the frequency of the low frequency carrier is used to represent a breathing cycle period of the user.

The low frequency carrier includes rising parts and falling parts, wherein the rising parts are used to represent one of the breathe-out and the breathe-in of a user, and the falling parts are used to represent the other one of the breathe-out and the breathe-in of the user. Meanwhile, it is able to real-timely provide at least one of the breathing cycle period, the breathe-out and the breathe-in to the user for reference or suggesting the user to adjust the breathing pattern and/or the breathing depth.

As mentioned above, a complete PPG waveform includes a mixed effect of the systole and pressures from blood vessels. In the present disclosure, a volume variation of blood vessels is detected by optical measurements to obtain said PPG signals.

As mentioned above, it is possible to use a PPG signal to indicate a frequency of the heart circulation. As the PPG signal is to detect a volume variation of blood vessels and all blood vessels in the human body are connected together, related information of a breathing depth and a breathing cycle period are obtainable from analyzed signals through analyzing the PPG signal.

For example, when a breathe-in occurs, muscular exertion squeezes blood vessels and causes the value of a PPG signal to rise up; on the contrary, when a breathe-out occurs, muscle relaxation causes the value of a PPG signal to fall down. A breathing frequency of the breathing system of a user is identifiable by analyzing the rising period and/or the falling period of the PPG signal.

In addition, by comparing with the user's activity, it is possible to arrange a breath detection system to output a prompt to direct a user how to adjust breaths. To be more precisely, it is able to suggest the user to adjust a breathing frequency, and a depth and speed of breathe-in and/or breathe-out. For example, when a user has the hyperventilation due to nervousness, it is able to suggest the user to relax from an equipment which is connected to the detected PPG signal; or when a user breathes too fast or too slow during exercising, it is able to suggest the user to adjust the breath pattern to match the current exercise strength. It is able to suggest the user by an auditory prompt such as a voice or music through a user's earphone, by a visual prompt through a user's portable device, or by body sensing, e.g., the vibration.

One embodiment of obtaining the breathe-in, the breathe-out and the breathing cycle period related to the user's breathing from a PPG signal is illustrated hereinafter.

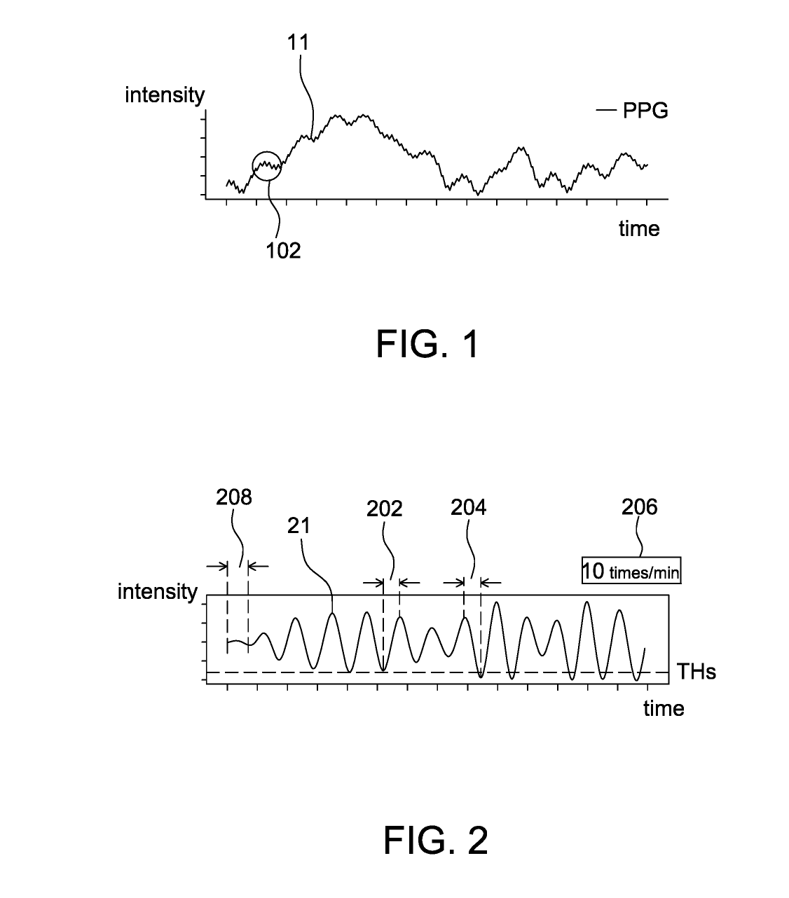

Firstly, a PPG signal is obtained by a breath detection device. As shown in FIG. 1, a high frequency part 102 of the PPG signal 11 indicates a frequency of the heart circulation. A low frequency carrier of the PPG signal 11 is then identified to determine one corresponding low frequency carrier signal 21, which has a low frequency capable of being used to indicate a breathing cycle period of a user, as shown in FIG. 2. Compared with FIG. 1, it is seen that there is a relationship between a variation speed of the low frequency carrier signal 21 in FIG. 2 and a carrier of the PPG signal in FIG. 1.

In one embodiment of the present disclosure, the breath detection device is further able to identify a rising part 202 and a falling part 204 of the low frequency carrier signal 21. As shown in one embodiment of FIG. 2, the rising part 202 represents a breathe-in and the falling part 204 represents a breathe-out. In other embodiments, due to the different processing of the obtained signal, it is possible that the rising part 202 represents a breathe-out and the falling part 204 represents a breathe-in. After obtaining the above information, it is able to real-timely output at least one of the breathing cycle period, the breathe-in and the breathe-out, and to suggest a user to adjust the whole breathing frequency or at least one of the breathe-in and the breathe-out. It is seen from FIG. 2 that high points and low points of the PPG signal in FIG. 1 do not exactly correspond to peaks and valleys of the low frequency carrier signal 21 in FIG. 2.

To be more precisely, FIG. 2 shows that each breath of a user is not exactly the same. Perhaps the frequency of breaths may be maintained almost the same, but the depth (e.g., amplitude) of the breathe-out and the breathe-in still changes. A user is hardly conscious of this change by him/herself in daily life. Therefore, by using the breath detection device in the embodiment of the present disclosure, it is able to help the user to understand his/her physiological states more, and achieve the effect of self-adjustment.

The present disclosure is also able to record user's breathing states for a long period of time to provide statistical data to the user as a reference for the self-adjustment, and it is possible to further determine thresholds according to said statistical data.

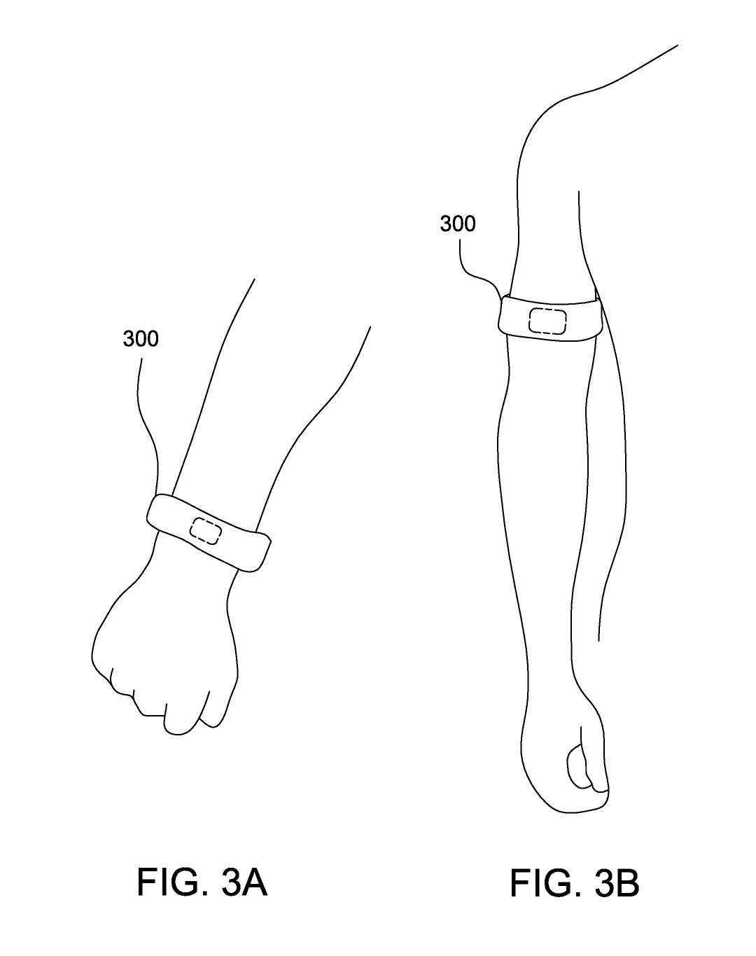

Please referring to FIGS. 3A and 3B, they are usage states of a breath detection device according to some embodiments of the present disclosure. The breath detection device 300 analyzes and displays the variation of a user's breathing signal changed with time, as shown in FIG. 2, by detecting a PPG signal of the user's skin tissues. Accordingly, the breath detection device 300 is able to be arranged at any suitable location to detect the PPG signal, e.g., setting on the user's wrist (FIG. 3A) or the user's arm (FIG. 3B), but not limited thereto. In another embodiment, the breath detection device 300 is integrated in a portable electronic device or a wearable electronic device, e.g., a bracelet, an armband, a ring, a foot ring, a foot bracelet, a cell phone, an earphone, a headphone and a personal digital assistant (PDA) which contacts at least a part of skin surface of a user. In addition, the breath detection device 300 is able to be coupled to a medical device, a home appliance, a vehicle, a security system in a wired or wireless way. Preferably, the one connected with the breath detection device 300 includes a display device to real-timely display a detection result of the breath detection device 300, e.g., directly displaying the low frequency carrier signal 21 as shown in FIG. 2.

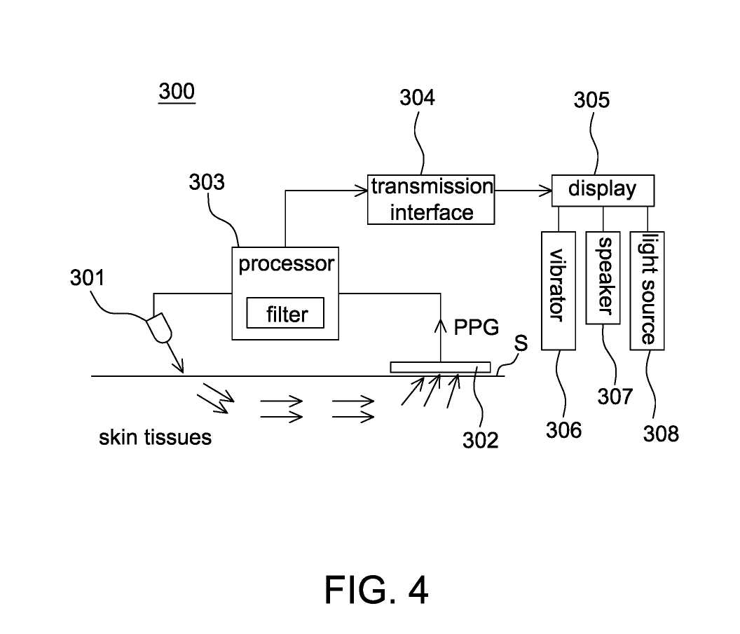

Please referring to FIG. 4, it is a schematic block diagram of a breath detection device 300 according to one embodiment of the present disclosure. The breath detection device 300 includes a light source 301, a light sensor 302 and a processor 303. In some embodiments, the breath detection device 300 further includes a display device 305 configured to display the detection result of the breath detection device 300. In some embodiments, the breath detection device 300 further includes a transmission interface 304 coupled to an external display device 305 in a wired or wireless manner to output the detection result (e.g., low frequency carrier signal 21) of the breath detection device 300 to the display device 305 to be real-timely displayed. In other words, the display device 305 may or may not be included in the breath detection device 300 depending on different applications. The display device 305 is, for example, a liquid-crystal display (LCD), a plasma display panel (PDP), an organic light-emitting diode (OLED) display or a projector for displaying images without particular limitations as long as it is able to display the low frequency carrier signal 21 as shown in FIG. 2 on a screen.

The light source 301 is, for example, a light emitting diode or a laser diode, configured to emit light adapted to penetrate and be absorbed by skin tissues. For example, a wavelength of light emitted by the light source is about 610 nm or 910 nm, but not limited thereto. The light source 301 illuminates a skin surface S to allow light to pass through skin tissues under the skin surface S. Preferably, the breath detection device 300 includes a transparent surface to be attached to the skin surface S in operation and for protecting the light source 301, and the light source 301 is arranged at an inner side of the transparent surface. The transparent surface is made of, e.g., plastic or glass without particular limitations.

In some embodiments, when the breath detection device 300 also detects the blood oxygenation, the breath detection device 300 includes two light sources to respectively emit different wavelengths of light, wherein the method of detecting the blood oxygenation may be referred to U.S. application Ser. No. 13/614,999 assigned to the same assignee of the present application, and the full disclosure of which is incorporated herein by reference.

The light sensor 302 is, for example, a photodiode or an image sensor array, e.g., a CMOS sensor array, and configured to detect ejected light emitted from the skin tissues to generate a PPG signal, as shown in FIG. 1 for example. The method of detecting and outputting a PPG signal by a photodiode is known to the art and thus details thereof are not described herein. The present disclosure is to identify breathing signals according to the detected PPG signal. The method of detecting a three dimensional physiology distribution by an image sensor array may be referred to U.S. application Ser. No. 14/955,463 assigned to the same assignee of the present application, and the full disclosure of which is incorporated here by reference. Similarly, the light sensor 302 is arranged inside of the transparent surface.

The processor 303 is, for example, a microcontroller (MCU), a central processing unit (CPU) or an application specific integrated circuit (ASIC), which is electrically coupled to the light source 301 and the light sensor 302, and configured to control the light source 301 and the light sensor 302 to operate correspondingly. The processor 303 acquires a low frequency carrier (e.g., the low frequency carrier signal 21 shown in FIG. 2) of the PPG signal (as shown in FIG. 1 for example) as a breathing signal, wherein said acquiring is implemented by software and/or hardware without particular limitations. For example, the processor 303 acquires the low frequency carrier signal 21 from the PPG signal by a digital band pass filter. Generally, a user's respiration rate is lower than 15 times per minute, so a pass band of the digital band pass filter is preferably lower than 0.25 Hz. It is appreciated that the pass band of the digital band pass filter is set according to the operation situation of the breath detection device 300 without particular limitations.

The transmission interface 304 outputs the breathing signal in a wired or wireless way, e.g., outputting data of the breathing signal at a predetermined frequency to a display device 305, wherein said wired and wireless transmission techniques are known to the art and thus details thereof are not described herein. It is appreciated that when the breath detection device 300 also includes the display device 305, the transmission interface 304 is not implemented or the transmission interface 304 is arranged inside the breath detection device 300 between the processor 303 and the display device 305.

The display device 305 real-timely displays a variation curve (i.e. the low frequency carrier signal 21) of the breathing signal changed with time as shown in FIG. 2. In addition, the processor 303 further calculates an intensity threshold THs correlated to the breathing signal (as shown in FIG. 2), a rising part 202, a falling part 204 and a frequency value 206, and sends the values and data to the display device 305 directly or via the transmission interface 304 to be displayed thereon. For example, lines, numbers or graphics are shown on a screen of the display device 305 to mark the intensity threshold THs, the rising part 202, the falling part 204 and the frequency value 206 to allow a user to easily observe his/her breathing states from the display device 305.

Different from conventional breath detection devices, the breath detection device 300 of the present disclosure is able to real-timely display a user's breathing state. In other words, as the breath detection device 300 analyzes a PPG signal detected by the light sensor 302 to acquire a breathing signal, when the processor 303 receives the PPG signal, the processor 303 starts to analyze and output the breathing signal to the display device 305 to be displayed thereon. Accordingly, although an initial stage of the breathing signal displayed by the display device 305 includes a convergence time 208 (e.g., as shown in FIG. 2), a time interval of the convergence time 208 is determined by the digital filter being used. The breathing signal is displayed normally after the convergence time 208. Generally, the convergence time is not long and lower than several seconds.

In addition, to improve the user experience, the breath detection device 300 further includes a prompt device (e.g., display device 305) to output a prompt signal according to a comparison result of comparing detected values, e.g., an intensity, an average intensity, a rising part, a falling part and/or a frequency, of the variation curve with at least one threshold, wherein the prompt signal is, e.g., a vibration signal, a light signal, an audio signal and/or an image signal without particular limitations as long as the user can be informed.

The breath detection device 300 of the present disclosure is applicable to the breathing control.

For example, when a user's breathing depth does not reach or exceeds a threshold, the prompt device 305 outputs a prompt signal. In one embodiment of the present disclosure, the intensity (i.e. amplitude) or average intensity of the variation curve of the breathing signal is used to represent a user's breathing depth, i.e. the higher the intensity, the longer the user's breathing; on the contrary, the lower the intensity, the shorter the user's breathing.

For example, when a user's breathing time does not reach or exceeds a threshold, the prompt device 305 outputs a prompt signal. In one embodiment of the present disclosure, the rising part 202 of the variation curve of the breathing signal is used to represent one of a breathing in state and the breathing out state of a user, and the falling part 204 of the variation curve of the breathing signal is used to represent the other one of the breathing in state and the breathing out state of the user, i.e. the longer the rising part 202 and the falling part 204, the longer the user's breathing time; on the contrary, the shorter the rising part 202 and the falling part 204, the shorter the user's breathing time.

For example, when a user's breathing frequency does not reach or exceeds a threshold, the prompt device 305 outputs a prompt signal. In one embodiment of the present disclosure, the frequency is used to represent a respiration rate of a user, e.g., displayed by a frequency value 206 together with the breathing signal (i.e. the low frequency carrier signal 21) on a display screen. In this embodiment, the processor 303 is able to calculate the breathing frequency according to one rising part 202 and one falling part 204 (e.g., calculating a reciprocal of a sum of interval of the rising part 202 and the falling part 204), and it is not necessary to accumulate count values for one minute.

The indicating method of the prompt signal is determined according to different applications.

For example, the display device 305 may also be used as the prompt device. When the detected values exceed or do not reach the threshold, the processor 303 provides image signals to the display device 305 to make the display device 305 display the prompt, e.g., by words, graphs, and/or brightness, etc.

For example, the breath detection device 300 further includes a vibrator 306 used as the prompt device. When the detected values exceed or do not reach the threshold, the processor 303 provides vibration signals to the vibrator 306 to make the vibrator 306 generate vibrations to hint the user.

For example, the breath detection device 300 further includes a speaker 307 used as the prompt device. When the detected values exceed or do not reach the threshold, the processor 303 provides voice signals to the speaker 307 to make the speaker 307 generate sounds to hint the user.

For example, the breath detection device 300 further includes a warning light source 308 used as the prompt device. When the detected values exceed or do not reach the threshold, the processor 303 provides optical signals to the warning light source 308 make the warning light source 308 illuminate light to hint the user.

In some embodiments, the processor 303 includes, for example, a learning algorithm (e.g., implemented by software and/or hardware), and the above thresholds (e.g., intensity threshold, time threshold and frequency threshold, but not limited thereto) are determined according to the user's history records. Information related to the history records is stored in, for example, a non-volatile memory.

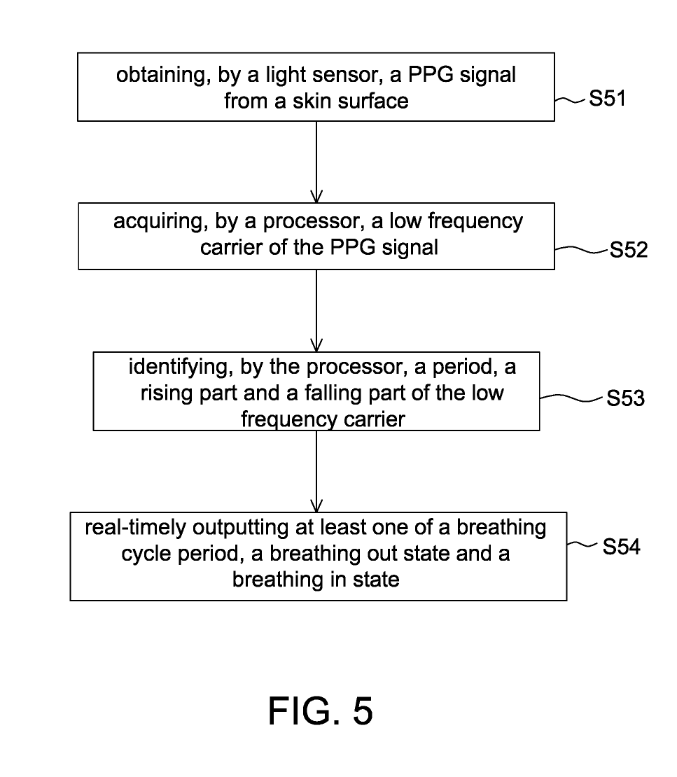

Please referring to FIG. 5, it is a flow chart of an operating method of a breath detection device according to one embodiment of the present disclosure, which includes the steps of: obtaining, by a light sensor, a PPG signal from a skin surface (step S51); acquiring, by a processor, a low frequency carrier of the PPG signal (step S52); identifying, by the processor, a period, a rising part and a falling part of the low frequency carrier (step S53), and real-timely outputting at least one of a breathing cycle period, a breathing out state and a breathing in state (step S54).

Step S51: The breath detection device 300 is preferably fixed with respect to a skin surface S in operation such that a PPG signal detected by the light sensor 302 is not affected by noises due to movement. In addition, the processor 303 further built-in with an algorithm for eliminating the noises in PPG signals caused by the movement, wherein the method of eliminating motion noises may be referred to U.S. application Ser. No. 13/614,999 assigned to the same assignee of the present application, and the full disclosure of which is incorporated herein by reference.

Step S52: The processor 303 starts to acquire a low frequency carrier signal 21 (as shown in FIG. 2) from a PPG signal right after receiving the PPG signal from the light sensor 302. In one embodiment, the processor 303 acquires the low frequency carrier signal 21 from the PPG signal using a digital band pass filter.

Step S53: After the processor 303 obtains the low frequency carrier signal 21, the processor 303 real-timely identifies a period, a rising part 202 and a falling part 204 of the low frequency carrier signal 21, wherein the period is used to indicate a user's breathing cycle period (e.g., including a rising part 202 and a falling part 204 adjacent to each other); the rising part 202 is used to indicate one of the user's breathe-in and breathe-out; and the falling part 204 is used to indicate the other one of the user's breathe-in and breathe-out. As mentioned above, in this embodiment the breath detection device 300 (or the processor 303) calculates a respiration rate of a user according to one breathing cycle period.

Step S54: Next, the processor 303 outputs at least one of the breathing cycle period, a breathing out state of the breathe-out and a breathing in state of the breathe-in to the display device 305 to be real-timely displayed thereon. In one embodiment, the display device 305 displays a variation curve of the low frequency carrier signal 21 changed with time such that the breathing cycle period, the breathing out state and the breathing in state are displayed at the same time. In another embodiment, the display device 305 displays values of the breathing cycle period, the breathing out state and the breathing in state instead of displaying the variation curve. In another embodiment, the display device 305 displays both of a variation curve of the low frequency carrier signal 21 with time as well as values of the breathing cycle period, the breathing out state and the breathing in state. Furthermore, the display device 305 further shows at least one of an intensity threshold mark, a rising part mark, a falling part mark with lines, characters or graphs to help a user to easily read information.

It should be mentioned that although the above embodiments take the reflective optical breath detection device as an example for illustration, it is only intended to illustrate but not to limit the present disclosure. In other embodiments, the breath detection device is a transmissive optical device in which disposed positions of the light source and the light sensor are different from the above embodiments but the sensing theory is not changed, and thus details thereof are not repeated herein.

As mentioned above, conventional breath detection devices are not able to real-timely display the user's breathing states such that applications thereof are limited. Therefore, the present disclosure further provides a breath detection device (as shown in FIG. 4) and an operating method thereof (as shown in FIG. 5) that real-timely calculate and display the user's breathing states and are not necessary to obtain a respiration rate by counting a plurality of breaths within a predetermined period. In addition, the breath detection device of the present disclosure is further able to help a user to adjust his/her breathing states by a prompting mechanism to effectively enhance the user experience and applicable ranges.

Although the disclosure has been explained in relation to its preferred embodiment, it is not used to limit the disclosure. It is to be understood that many other possible modifications and variations can be made by those skilled in the art without departing from the spirit and scope of the disclosure as hereinafter claimed.

What is claimed is:

1. A breath detection device comprising:
a light source configured to illuminate a skin surface to allow light to pass through skin tissues under the skin surface;
a light sensor configured to detect ejected light from the skin tissues to generate a photoplethysmography (PPG) signal; and
a processor configured to acquire a low frequency carrier of the PPG signal as a breathing signal.

2. The breath detection device as claimed in claim 1, further comprising a transmission interface configured to output the breathing signal.

3. The breath detection device as claimed in claim 2, wherein the transmission interface is configured to send the breathing signal to a display device, and the display device is configured to real-timely display a variation curve of the breathing signal with time.

4. The breath detection device as claimed in claim 3, wherein the transmission interface is further configured to send an intensity threshold mark associated with the breathing signal to the display device to be displayed thereon.

5. The breath detection device as claimed in claim 3, wherein the transmission interface is further configured to send a rising part mark and a falling part mark associated with the breathing signal to the display device to be displayed thereon.

6. The breath detection device as claimed in claim 3, wherein the transmission interface is further configured to send a frequency value mark associated with the breathing signal to the display device to be displayed thereon.

7. The breath detection device as claimed in claim 1, wherein the processor is configured to acquire the low frequency carrier from the PPG signal using a digital band pass filter.

8. The breath detection device as claimed in claim 7, wherein a pass band of the digital band pass filter is lower than 0.25 Hz.

9. The breath detection device as claimed in claim 1, wherein the light sensor is a photodiode or an image sensor array.

10. The breath detection device as claimed in claim 1, wherein a wavelength of light emitted by the light source is 610 nm or 910 nm.

11. The breath detection device as claimed in claim 1, wherein the breath detection device is integrated with a portable electronic device or a wearable electronic device.

12. A breath detection device comprising:
a light source configured to illuminate a skin surface to allow light to pass through skin tissues under the skin surface;
a light sensor configured to detect ejected light from the skin tissues to generate a photoplethysmography (PPG) signal;
a processor configured to acquire a low frequency carrier of the PPG signal as a breathing signal;
a display device configured to real-timely display a variation curve of the breathing signal with time; and
a prompt device configured to generate a prompt signal according to a comparison result of comparing at least one of intensity, a rising part, a falling part and a frequency of the variation curve with at least one threshold.

13. The breath detection device as claimed in claim 12, wherein the prompt signal is at least one of a vibration signal, a light signal, an audio signal and an image signal.

14. The breath detection device as claimed in claim 12, wherein
the intensity is configured to represent a breathing depth of a user;
the rising part is configured to represent one of a breathing out state and a breathing in state of the user,
the falling part is configured to represent the other one of the breathing out state and the breathing in state of the user; and
the frequency is configured to represent a respiration rate of the user.

15. The breath detection device as claimed in claim 12, wherein the processor is configured to acquire the low frequency carrier from the PPG signal using a digital band pass filter.

16. The breath detection device as claimed in claim 12, wherein the at least one threshold is determined according to a user's history record.

17. The breath detection device as claimed in claim 12, wherein the breath detection device is integrated with a portable electronic device or a wearable electronic device.

18. An operating method of a breath detection device, the breath detection device comprising a light sensor and a processor, the operating method comprising:
obtaining, by the light sensor, a photoplethysmography (PPG) signal from a skin surface;
acquiring, by the processor, a low frequency carrier of the PPG signal;
identifying, by the processor, a period, a rising part and a falling part of the low frequency carrier, wherein the period represents a breathing cycle period of a user, the rising part represents one of a breathe-out and a breathe-in of the user, and the falling part represents the other one of the breathe-out and the breathe-in of the user; and
real-timely outputting at least one of the breathing cycle period, a breathing out state of the breathe-out and a breathing in state of the breathe-in.

19. The operating method as claimed in claim 18, wherein the processor acquires the low frequency carrier from the PPG signal using a digital band pass filter.

20. The operating method as claimed in claim 18, further comprising:
displaying, by a display device, a variation curve of the low frequency carrier with time to display the breathing cycle period, the breathing out state and the breathing in state at the same time.
